(19) United States
(12) Patent Application Publication
(10) Pub. No.: US 2010/0145209 A1
(43) Pub. Date: Jun. 10, 2010

(54) APPARATUS FOR MEASURING AMOUNT OF EXERCISE, SYSTEM FOR MEASURING AMOUNT OF EXERCISE, AND METHOD FOR MEASURING AMOUNT OF EXERCISE

(76) Inventors: In Bum LEE, Daejeon (KR); Seung Chul SHIN, Daejeon (KR); Yong Won JANG, Daejeon (KR); Yoon Seon SONG, Daejeon (KR); Seung Hwan KIM, Daejeon (KR); Jeong Won LEE, Daejeon (KR); Seon Hee PARK, Daejeon (KR)

Correspondence Address: LADAS & PARRY LLP, 224 SOUTH MICHIGAN AVENUE, SUITE 1600, CHICAGO, IL, 60604, US

(21) Appl. No.: 12/505,585

(22) Filed: Jul. 20, 2009

(30) Foreign Application Priority Data
Dec. 8, 2008 (KR) 10-2008-0124281

(51) Int. Cl. A61B 5/0402; A61B 5/11

(52) U.S. Cl. 600/519; 600/595

(57) ABSTRACT

An apparatus for measuring the amount of exercise, a system for measuring the amount of exercise, and a method for measuring the amount of exercise are disclosed. The apparatus for measuring the amount of exercise includes: an exercise amount measuring unit that measures the amount of exercise of a user; a controller that generates information about exercise of each period from the amounts of exercise of the user measured at every predefined period; a memory unit that sequentially stores the information about the exercise of each period; and a communication unit that transmits the stored information about the exercise of each period to the exterior.

### CROSS-REFERENCE TO RELATED APPLICATIONS

This application claims the priority of Korean Patent Application No. 2008-0124281 filed on Dec. 8, 2008, in the Korean Intellectual Property Office, the disclosure of which is incorporated herein by reference.

### BACKGROUND OF THE INVENTION

1. Field of the Invention

The present invention relates to an apparatus for measuring the amount of exercise, a system for measuring the amount of exercise, and a method for measuring the amount of exercise, and more particularly, to an apparatus for measuring the amount of a user's exercise, a system for measuring the amount of a user's exercise, and a method for measuring the amount of a user's exercise capable of measuring the amount of a user's exercise and displaying information about the measured amount of exercise on a different portable device.

2. Description of the Related Art

As the percentage of the population who are obese and overweight is increasing as a result of supernutrition and lack of exercise, the necessity of managing health care for modern people is growing. In addition, recently, even people who are in good shape are paying greater attention to their health, and the feasibility of ubiquitous health care enables such interest to be substantialized in line with the development of technology and social requirements.

To this end, diverse items of equipment for calculating consumed calories based on the amount of exercise and metabolism activity (operation) of users by using gravity sensors, body temperature sensors, electrocardiogram sensors and the like, are under development. Users of these devices tend to want to measure the amount of exercise and consumed calories in the long term, beyond those interested in short term measurements, and also increasingly tend to want management of exercise by specialists based on measured data.

However, in order to perform such functions, the related art apparatus for measuring the amount of exercise must include a display unit for displaying measurement result of the amount of exercise, alarm messages, and the like, for user recognition, a user interface for controlling the apparatus such as controlling the image of the result displayed on the display unit, a real time clock (RTC) for date information, and the like. Thus, the presence of the display unit, the user interface, and the RTC make the exercise amount measurement apparatus bigger and increase the costs of the apparatus as a result of the increase in the development and material costs.

Namely, the related art exercise amount measurement apparatus has a complicated structure, is large in size, and incurs high developmental and fabrication costs.

### SUMMARY OF THE INVENTION

An aspect of the present invention provides an apparatus for measuring the amount of exercise, a system for measuring the amount of exercise, and a method for measuring the amount of exercise capable of providing multiple functions yet incurring low development and fabrication costs through a simple structure and small size.

According to an aspect of the present invention, there is provided an apparatus for measuring the amount of exercise including: an exercise amount measuring unit that measures the amount of exercise of a user; a controller that generates information about exercise of each period from the amounts of exercise of the user measured at every predefined period; a memory unit that sequentially stores the information about the exercise of each period; and a communication unit that transmits the stored information about the exercise of each period to the exterior.

The exercise amount measurement apparatus may further include a display unit that displays the information about the exercise of each period.

The exercise amount measuring unit may include a three-axis gravity sensor that detects the amount of an activity mass according to the exercise of the user. In this case, the memory unit may store an activity mass-calorie consumption table indicating the correlation between the activity mass and consumed calories of the user measured by the three-axis gravity sensor, and the controller may measure consumed calories according to the activity mass of the user measured by the three-axis gravity sensor with reference to the activity mass-calorie consumption table. Also, the controller may obtain the variance of each period from a signal measured by the three-axis gravity sensor according to a predefined sampling frequency at every predefined period, determine the variance of each period as an activity mass, and calculate consumed calories from the activity mass with reference to the activity mass-calorie consumption table.

Instead, or in addition, the exercise amount measuring unit may include an electrocardiogram sensor that detects a heart rate of the user. In this case, the memory unit may store a heart rate-calorie consumption regression analysis result table according to a minimum heart rate and a maximum heart rate of the user which has been previously measured, and the controller may measure consumed calories according to the heart rate detected by the electrocardiogram sensor with reference to the heart rate-calorie consumption regression analysis result table. In addition, the controller may calculate a heart rate from a signal measured by the electrocardiogram sensor at every predefined period and calculate consumed calories from the heart rate with reference to the heart rate-calorie consumption regression analysis result table.

According to another aspect of the present invention, there is provided a system for measuring the amount of exercise including: an exercise amount measurement apparatus that measures the amount of exercise; and a portable device that analyzes information about exercise of each period stored in a memory of the exercise amount measurement apparatus, calculates consumed calories of the user, and outputs the calculated consumed calories, wherein the exercise amount measurement apparatus comprises: an exercise amount measuring unit that measures the amount of exercise of a user; a controller that generates information about exercise of each period from the amounts of exercise of the user measured at every predefined period; a memory unit that sequentially stores the information about the exercise of each period; and a communication unit that transmits the stored information about the exercise of each period to the portable device.

The portable device may include one of a mobile communication terminal, a personal digital assistant (PDA), a portable multimedia player (PMP), an MP3 player, and a notebook computer.

The portable device may calculate time information from the exercise information of each period and exercise information corresponding to the time information by using current time information of the portable device, and display the calculated time information and exercise information.

The exercise amount management system may further include: an exercise management server that manages the amount of exercise of the user. In this case, the portable device may calculate time information from the exercise information of each period and exercise information corresponding to the time information from the exercise information of each period by using current time information of the portable device, and supply the calculated time information and exercise information to the exercise management server.

The portable device may supply personal identification information of the user to the exercise management server, and the exercise management server may store and process the time information and the exercise information of each user by using the personal identification information.

According to still another aspect of the present invention, there is provided a method for measuring the amount of exercise including: sequentially receiving exercise information of each period obtained by measuring the amount of exercise of a user at every predefined period; calculating time information according to the exercise information of each period and exercise information corresponding to the time information by using the received exercise information of each period and current time information; and displaying the time information and the exercise information.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a schematic conceptual view of an exercise amount management system according to an exemplary embodiment of the present invention;

FIG. 2 is a schematic block diagram showing an internal configuration of an exercise amount measurement apparatus of FIG. 1; and

FIG. 3 is a flow chart illustrating an exercise amount measurement method according to an exemplary embodiment of the present invention.

### DETAILED DESCRIPTION OF THE PREFERRED EMBODIMENT

Exemplary embodiments of the present invention will now be described in detail with reference to the accompanying drawings. The invention may however be embodied in many different forms and should not be construed as limited to the embodiments set forth herein. Rather, these embodiments are provided so that this disclosure will be thorough and complete, and will fully convey the scope of the invention to those skilled in the art. In the drawings, the shapes and dimensions may be exaggerated for clarity, and the same reference numerals will be used throughout to designate the same or like components.

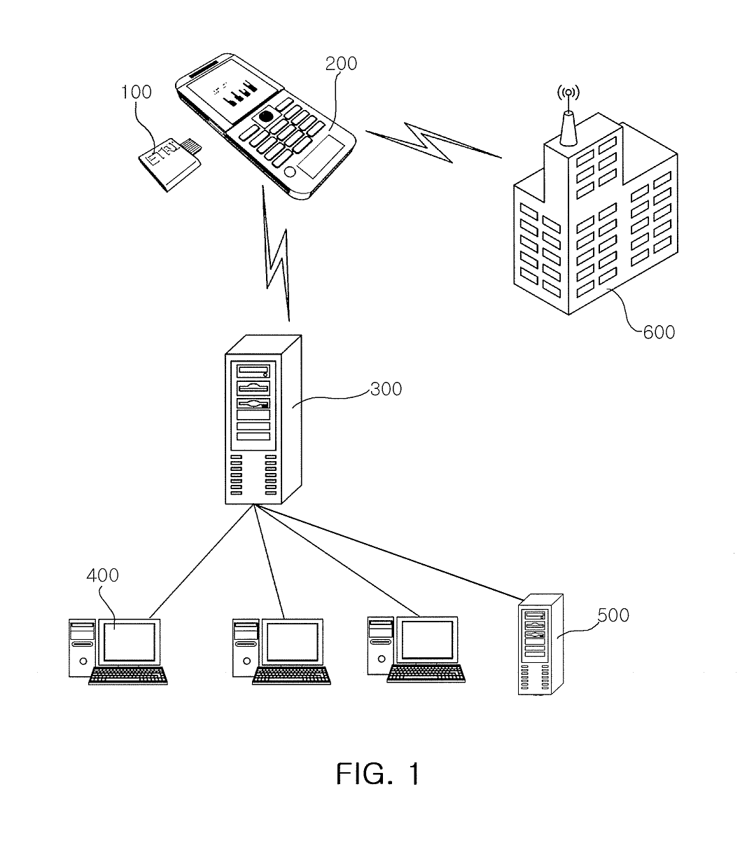

FIG. 1 is a schematic conceptual view of an exercise amount management system according to an exemplary embodiment of the present invention. As shown in FIG. 1, the exercise amount management system according to an exemplary embodiment of the present invention may include an exercise amount measurement apparatus 100, a portable device 200, an exercise amount data management server 300, a user terminal 400, a health management server 500, and an Internet data center 600.

The exercise amount measurement apparatus 100 is carried around by a user and measures and stores the amount of exercise of the user and consumed calories. A detailed configuration of the exercise amount measurement apparatus 100 will be described in more detail later. When the exercise amount measurement apparatus 100 is connected to the portable device 200, the user may request information about the amount of exercise or consumed calories (specifically, exercise information of each period as to be described later) through the exercise amount measurement apparatus 100.

The portable device 200 may include one of a mobile communication terminal, a PDA, a notebook computer, a PMP, and an MP3 player. But, it is merely illustrative and the portable device according to an exemplary embodiment of the present invention is not limited thereto. When the portable device is a mobile communication terminal, the exercise amount measurement apparatus 100 may be connected to the mobile communication terminal 200 via a mobile communication terminal standard connection unit as shown in FIG. 1.

Hereinafter, it is assumed that the portable device 200 is the mobile communication terminal. The user may request exercise information such as data about the amount of exercise and consumed calories (specifically, exercise information of each period as to be described later) through the exercise amount measurement apparatus 100 by using the user interface (e.g., a keypad) of the mobile communication terminal 200.

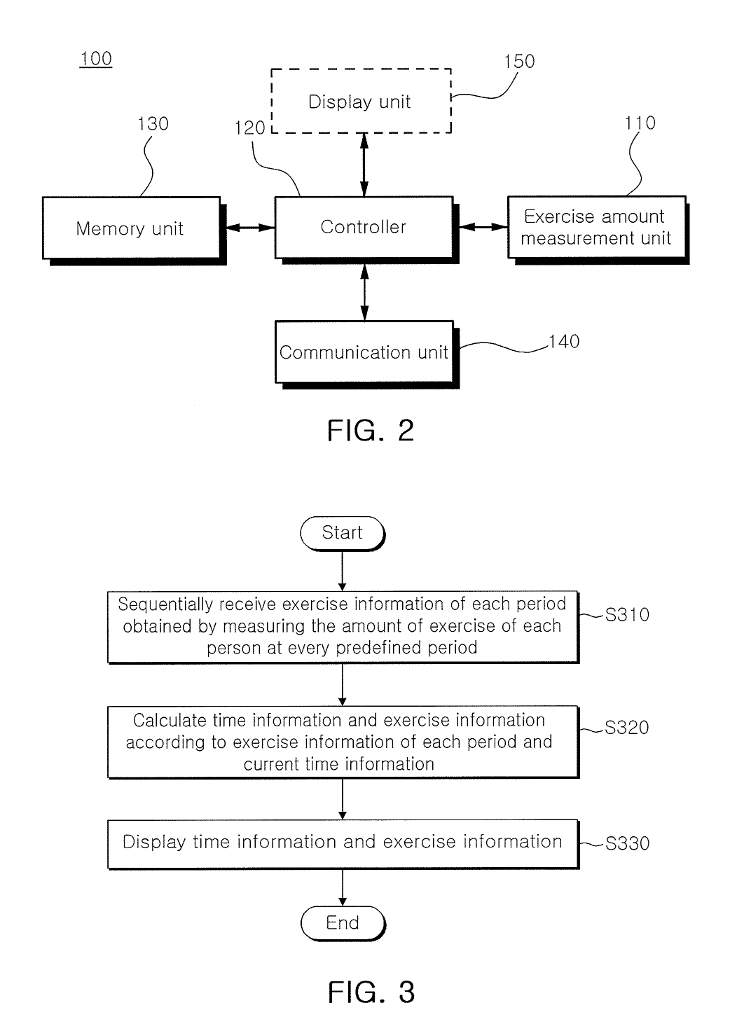

When the exercise amount measurement apparatus 100 receives the request for transmission of the exercise information from the mobile communication terminal 200, the exercise amount measurement apparatus 100 transmits the exercise information to the mobile communication terminal 200 via a certain communication means (e.g., a communication unit 140 in FIG. 2 (to be described)). The mobile communication terminal may process the received exercise information and display the processed exercise information on its display unit, or may transmit the processed exercise information to the exercise amount data management server 300. In this case, in transmitting the exercise information, the mobile communication terminal 200 may also transmit personal identification information of the user stored in the mobile communication terminal 200 or personally (directly) inputted by the user.

The exercise amount data management server 300 checks the identity of the user of the mobile communication terminal 200 by using the received personal identification information and stores the received exercise information in a corresponding account of the user.

Meanwhile, before the user connects his mobile communication terminal 200 to the exercise amount measurement apparatus 100, the user may download a program (i.e., a mobile communication terminal application) providing a communication function with the exercise amount measurement apparatus 100, a display function of the exercise information, and the like, and install the downloaded program in the mobile communication terminal 200. Once the program is installed initially one time, it may not need to be installed again. In a different embodiment, when the program needs updating, an updated program may be downloaded via the Internet data center 600 so as to be installed in the mobile communication terminal 200.

The user of the exercise amount measurement apparatus 100 may request his exercise information to the exercise amount data management server 300 by using the mobile communication terminal 200 to check the result, or alternatively, the user may access the exercise amount data management server 300 by using the user terminal 400, namely, a communication tool such as a personal computer (PC), to check the result.

Also, the permitted health management server 500 may access the exercise amount data management server 300 to read the exercise information, based on which the health management server 500 may perform exercise and health management of each person.

In FIG. 1, the health management server 500 and the exercise amount data management server 300 are separately illustrated, but they may be the same device or separate devices. The health management server 500 and the exercise amount data management server 300 may be combined to configure an exercise management server according to an exemplary embodiment of the present invention.

The configuration of the exercise amount measurement apparatus 100 will now be described in detail with reference to FIG. 2. FIG. 2 is a schematic block diagram showing the internal configuration of the exercise amount measurement apparatus of FIG. 1. The exercise amount measurement apparatus 100 includes an exercise amount measurement unit 110, a controller 120, a memory unit 130, and the communication unit 140. The exercise amount measurement apparatus 100 according to an exemplary embodiment of the present invention may further include a display unit 150.

The exercise amount measurement unit 110 may include one of a three-axis gravity sensor, a body heat sensor, and an electrocardiogram sensor, and their combinations, and detects a signal for measuring the amount of exercise and consumed calories of the user who wears the exercise amount measurement apparatus 100. One or more sensors may be simultaneously used to calculate various types of amounts of exercises and consumed calories.

The exercise amount measurement unit 110 may supply signals detected from the sensors to the controller 120, or alternatively, the electrical signals detected from the sensors may be periodically binarized through A/D conversion so as to be transferred to the controller 120. To this end, an A/D converter (not shown) for periodically binarizing the electrical signals may be required, and it may be included in the controller 120 or in the exercise amount measurement unit 110. Also, in a different embodiment, the A/D converter may be separately configured to binarize the electrical signals transferred from the exercise amount measurement unit 110 to the controller 120.

The controller 120 calculates the amount of exercise and consumed calories by processing information about the weight, height, age, gender, etc., of the user and the signal supplied from the exercise amount measurement unit 110. To this end, the weight, height, age, gender, etc., of the user may be previously inputted, and in this case, these data may be stored in the memory unit 130.

The data regarding the calculated amount of exercise and consumed calories are stored in the memory unit 130. When the controller 120 receives a request for transmission of the exercise information stored in the memory unit 130 via the communication unit 140, it reads the exercise information from the memory unit 130 and transmits it to a connection unit 160 via the communication unit 140.

When the portable device 200 is the mobile communication terminal, the communication unit 140 may be fabricated in the form of a standard input/output terminal of the mobile communication terminal so as to be connected to the mobile communication terminal. In addition, a dongle may be used to be connected to a USB terminal of the PC or the like. When the portable device 200 supports a near-field communication network, the communication unit 140 may use a radio communication scheme such as Bluetooth™, Zigbee, and the like. Various radio communication schemes are illustrated in the exemplary embodiments of the present invention, but the present invention is not limited thereto and any wired/wireless communication scheme may be used so long as it can transmit and receive data.

In the exemplary embodiment of the present invention, the exercise amount measurement apparatus 100 includes the exercise amount measurement unit 110, the controller 120, the memory unit 130, and the communication unit 140, and may not include the display unit. The exercise information measured by the exercise amount measurement apparatus 100 is displayed on the display unit of the portable device 200 connected to the exercise amount measurement apparatus 100. Currently, mobile communication terminals, personal digital assistants (PDAs), notebook computers, portable multimedia players (PMPs), MP3 players, and the like, include a display unit and a user interface unit with high user convenience by themselves, so the exercise amount measurement apparatus 100 may make use of the display unit of the portable device 200 to thus have a simple structure.

In a different embodiment of the present invention, the exercise amount measurement apparatus 100 may not include a user command input unit or may include a minimum input unit. The minimum input unit may be, for example, a simple power ON/OFF button. Or, the exercise amount measurement apparatus 100 may not include such input unit.

In a different embodiment of the present invention, the exercise amount measurement apparatus 100 may not have a real time clock. In comparison, the related art exercise amount measurement apparatus includes a real time clock to store exercise information of the user by time zone and provide it. However, the exercise amount measurement apparatus 10c according to the exemplary embodiment of the present invention does not have such real time clock and simply calculate exercise information of each period and provide the same to the portable device 200. Calculation of the exercise information by time from the exercise information by period may be performed by a program installed in the mobile communication terminal 200.

Because the exercise amount measurement apparatus 100 does not include one or more of the display units, the user command input unit, and the real time clock, it has a simpler structure and smaller size than the related art exercise amount measurement apparatus. Thus obviously results in a reduction in the development costs and material costs of the exercise amount measurement apparatus 100.

The process of providing exercise information by time to the user according to cooperative operations between the exercise amount measurement apparatus 100 and the portable device 200 will now be described with reference to FIGS. 1 and 2.

A. Process of Calculating Exercise Information of Each Period by the Exercise Amount Measurement Apparatus 100 and Transmitting the Same to the Portable Device 200

(1) The exercise amount measurement unit 110 measures an exercise state of the user wearing the exercise amount measurement apparatus 100. The A/D converter periodically binarizes an electrical signal regarding the exercise state outputted from the exercise amount measurement unit 110 and transfers the same to the controller 120.

(2) The controller 120 calculates exercise information of each period by using the signal regarding the exercise state binarized at every certain period. This process will now be described in detail by taking the cases where the exercise amount measurement unit 110 includes the three-axis gravity sensor and the electrocardiogram sensor, respectively, as examples.

First, the case where the exercise amount measurement unit 110 includes the three-axis gravity sensor, the period is four seconds, and a sampling frequency is 20 Hz will now be described. In this case, the memory unit 130 includes an activity mass-calorie consumption table regarding consumed calories for each activity mass stored therein.

The controller 120 samples a signal regarding an exercise state which has been measured by the three-axis gravity sensor and binarized by the A/D converter at a frequency of 20 Hz, and obtains a variance of a value sampled at every four-second time interval. Namely, a single variance is obtained for every four seconds. The variance of each period is defined as an activity mass of the user. The controller 120 calculates consumed calories of the user corresponding to the activity mass with reference to the activity mass-calorie consumption table. Because the variance is obtained at every four seconds, the activity mass and the consumed calories are calculated in the cycle of four seconds. The activity mass or the consumed calories are one of exercise information provided to the user afterward, and in this exemplary embodiment, because the controller 120 calculates the exercise information by period, the exercise information may be called exercise information of each period. Four seconds and 20 Hz are merely illustrative. That is, the present invention is not limited thereto and any other periods and sampling frequencies may be used.

In a different embodiment, the case where the exercise amount measurement unit 110 includes the electrocardiogram sensor, the period is four seconds, the sampling frequency is 20 Hz will now be described. In this case, the memory unit 130 includes a heart rate-calorie consumption regression analysis result table according to a minimum heart rate and a maximum heart rate of the user which has been previously measured, stored therein. The controller 120 samples a signal regarding an exercise state which has been measured by the electrocardiogram sensor and binarized by the A/D converter at a frequency of 20 Hz, and calculates a heart rate from values sampled at every four-second time interval. Namely, a single heart rate is obtained at every four seconds. The controller 120 calculates consumed calories of the user corresponding to the heart rate with reference to the heart rate-calorie consumption regression analysis result table. Because the heart rate is obtained at every four seconds, the heart rate and consumed calories are calculated in the cycle of four seconds. The heart rate or the consumed calories are one of exercise information provided to the user afterward, and in this exemplary embodiment, because the controller 120 calculates the exercise information by period, the exercise information may be called exercise information of each period. Likewise as the three-axis gravity sensor, four seconds and 20 Hz are merely illustrative. That is, the present invention is not limited thereto and any other periods and sampling frequencies may be used.

(3) The controller 120 sequentially stores the calculated exercise information of each period in the memory unit 130.

(4) In a state that the communication unit 140 is connected to the portable device 200, the controller 120 transmits the exercise information of each period to the portable device 200 while maintaining the order of the exercise information stored in the memory unit 130.

According to this process, the exercise information of each period of the user can be transmitted from the exercise amount measurement apparatus 100 to the portable device 200.

B. Process of Displaying Exercise Information by Time on the Portable Device 200 and Transmitting the Exercise Information by Time to the Exercise Amount Data Management Server 300

(1) The portable device 200 provides control to store the exercise information of each period sequentially transmitted from the exercise amount measurement apparatus 100 in a memory of the portable device 200.

(2) The portable device 200 calculates time information and exercise information corresponding to the time information from the exercise information of each period stored in the memory by using current time information of the portable device 200.

With this process, for example, the case where exercise information of each period is an activity mass obtained at every four seconds and consumed calories corresponding to the activity mass will now be described in detail. As the exercise information of each period transmitted from the exercise amount measurement apparatus 100, consumed calories calculated for every four seconds are sequentially stored in the memory of the portable device 200. The portable device 200 reads the time information and the exercise information corresponding to the time information, namely, the consumed calories, from the stored exercise information of each period. Because the exercise information of each period are sequentially stored, time information at which the exercise information of each period was measured can be obtained by reversely counting storage intervals (i.e., the period of four seconds in the present embodiment) of the exercise information of each period. If the number of values of the stored consumed calories is 1,000, it could be interpreted such that the exercise information of each period have been stored at the intervals of four seconds from 4000 seconds before from a current time of the mobile communication terminal (i.e., 4×1000). Namely, although the exercise amount measurement apparatus 100 does not have a real time clock, the accurate time information at which the exercise information of each period have been measured can be known (obtained).

(3) The portable device 200 displays the calculated time information and exercise information on its display unit. Accordingly, the user can be provided with the exercise information such as the amount of exercise done by the user by time and corresponding consumed calories.

The operation of the portable device 200 as described above can be performed by a program installed in the terminal, and the program may be a program which has been basically installed in the portable device 200 or a program which has been installed in the portable device after being downloaded from the Internet data center 600. Also, the above-described program may be performed according to an exercise information transmission command, an exercise information display command, and the like, inputted via an input unit of the portable device 200 by the user.

The time information and the exercise information corresponding to the time information may be transmitted to the exercise amount data management server 300, so that the user terminal 400 or the health management center 500 may receive the time information and the corresponding exercise information from the exercise amount data management server 300. The configuration is the same as described above.

Various information stored in the memory unit 130 of the exercise amount measurement apparatus 100 may be those which have been transmitted from the portable device 200 to the exercise amount measurement apparatus 100 in a state that the exercise amount measurement apparatus 100 was connected to the portable device 200. For example, information regarding the weight, height, age, and gender of the user, the activity mass-calorie consumption table, or the like, are those transmitted from the portable device 200 and stored in the exercise amount measurement apparatus 100, after being inputted to the portable device 200 by the user or an allowed manager by using a user interface of the portable device 200 or after being transmitted to the portable device 200 from a device such as the user terminal 400, the exercise amount data management server 300, or the like.

A method for measuring the amount of exercise according to an exemplary embodiment of the present invention will now be described with reference to FIG. 3. FIG. 3 is a flow chart illustrating the operation of the exercise amount measurement method according to an exemplary embodiment of the present invention. As shown in FIG. 3, according to the exercise amount measurement method, first, exercise information of each period obtained by measuring the amount of exercise of each person (i.e., user) at every predefined period is sequentially received (S310). In this method, the method for measuring the amount of exercise of each person and the process of generating and storing exercise information of each period are the same as described above. Next, time information according to the exercise information of each period and exercise information corresponding to the time information are calculated by using the received exercise information of each period and current time information (S320). And then, the calculated time information and exercise information are displayed (S330) Steps S320 and S330 may be performed by the portable device 200 as illustrated in FIG. 1 and detailed performing process of the steps are the same as described above.

As set forth above, according to exemplary embodiments of the invention, because the function of the exercise amount measurement apparatus can be simplified by utilizing the elements already existing in the portable device and exercise information by time can be systematically provided to the user, the exercise amount measurement apparatus has a simpler configuration than that of the related art exercise amount measurement apparatus.

Also, because the exercise amount measurement apparatus has the simpler configuration, the development and fabrication costs, size, and weight of the exercise amount measurement apparatus can be reduced, thus enhancing portability of the portable device.

In addition, the system and method for providing exercise information according to the exemplary embodiments of the present invention allow a permitted outsider (e.g., an expert, specialist, etc) to easily search exercise information transmitted to an exercise data management server, the exercise management service by the expert can be provided.

While the present invention has been shown and described in connection with the exemplary embodiments, it will be apparent to those skilled in the art that modifications and variations can be made without departing from the spirit and scope of the invention as defined by the appended claims.

What is claimed is:

1. An apparatus for measuring the amount of exercise, the apparatus comprising:
an exercise amount measuring unit that measures the amount of exercise of a user;
a controller that generates information about exercise of each period from the amounts of exercise of the user measured at every predefined period;
a memory unit that sequentially stores the information about the exercise of each period; and
a communication unit that transmits the stored information about the exercise of each period to the exterior.

2. The apparatus of claim 1, further comprising:
a display unit that displays the information about the exercise of each period.

3. The apparatus of claim 1, wherein the exercise amount measuring unit comprises a three-axis gravity sensor that detects the amount of an activity mass according to the exercise of the user.

4. The apparatus of claim 3, wherein the memory unit stores an activity mass-calorie consumption table showing the correlation between the activity mass and consumed calories of the user, and the controller measures consumed calories according to the activity mass of the user measured by the three-axis gravity sensor with reference to the activity mass-calorie consumption table.

5. The apparatus of claim 4, wherein the controller obtains a variance of each period from a signal measured by the three-axis gravity sensor according to a predefined sampling frequency at every predefined period, determines the variance of each period as an activity mass, and calculates consumed calories from the activity mass with reference to the activity mass-calorie consumption table.

6. The apparatus of claim 1, wherein the exercise amount measuring unit comprises an electrocardiogram sensor that detects a heart rate of the user.

7. The apparatus of claim 6, wherein the memory unit stores a heart rate-calorie consumption regression analysis result table according to a minimum heart rate and a maximum heart rate of the user which has been previously measured, and the controller measures consumed calories according to the heart rate detected by the electrocardiogram sensor with reference to the heart rate-calorie consumption regression analysis result table.

8. The apparatus of claim 7, wherein the controller calculates a heart rate from a signal measured by the electrocardiogram sensor at every predefined period and calculates consumed calories from the heart rate with reference to the heart rate-calorie consumption regression analysis result table.

9. A system for measuring the amount of exercise comprising:
an exercise amount measurement apparatus that measures the amount of exercise; and
a portable device that analyzes information about exercise of each period stored in a memory of the exercise amount measurement apparatus, calculates consumed calories of the user, and outputs the calculated consumed calories,
wherein the exercise amount measurement apparatus comprises:
an exercise amount measuring unit that measures the amount of exercise of a user;
a controller that generates information about exercise of each period from the amounts of exercise of the user measured at every predefined period;
a memory unit that sequentially stores the information about the exercise of each period; and
a communication unit that transmits the stored information about the exercise of each period to the portable device.

10. The system of claim 9, wherein the portable device comprises one of a mobile communication terminal, a personal digital assistant (PDA), a portable multimedia player (PMP), an MP3 player, and a notebook computer.

11. The system of claim 9, wherein the portable device calculates time information and exercise information corresponding to the time information from the exercise information of each period by using current time information of the portable device, and displays the calculated time information and exercise information.

12. The system of claim 9, further comprising:
an exercise management server that manages the amount of exercise of the user, and
the portable device calculates time information and exercise information corresponding to the time information from the exercise information of each period by using current time information of the portable device, and supplies the calculated time information and exercise information to the exercise management server.

13. The system of claim 12, wherein the portable device supplies personal identification information of the user to the exercise management server, and the exercise management server stores and processes the time information and the exercise information of each user by using the personal identification information.

14. A method for measuring the amount of exercise, the method comprising:
sequentially receiving exercise information of each period obtained by measuring the amount of exercise of a user at every predefined period;
calculating time information according to the exercise information of each period and exercise information corresponding to the time information by using the received exercise information of each period and current time information; and
displaying the time information and the exercise information.
